(19) United States
(12) Patent Application Publication
(10) Pub. No.: US 2023/0154617 A1
(43) Pub. Date: May 18, 2023

(54) Method and System for Examining Health Conditions of an Animal

(71) Applicant: EquiVet Care, Inc., Chuluota, FL (US)

(72) Inventors: Hanna Lannes, Chuluota, FL (US); Edgar Munoz, Chuluota, FL (US); Jillian Costello Chavers, Ocala, FL (US); Ed Yeh, Palo Alto, CA (US); Jeremy Pascual, San Jose, CA (US); Keith Koo, Fremont, CA (US)

(21) Appl. No.: 17/988,969

(22) Filed: Nov. 17, 2022

Related U.S. Application Data
(60) Provisional application No. 63/280,325, filed on Nov. 17, 2021.

(51) Int. Cl. G16H 50/20; G16H 10/60; G16H 50/70; A61B 5/024

(52) U.S. Cl. CPC G16H 50/20; G16H 10/60; G16H 50/70; A61B 5/02405; A61B 2503/40

(57) ABSTRACT

A method for identifying causative ailments or conditions for health-related symptoms of an animal. Information from a pet profile database and symptoms are received. First-tier data options are selected by a user and an algorithm analyzes them using information from a medical knowledge database. Data options associated with each successive tier are determined by the algorithm, based on user-selected responses to data options of a prior tier and information from the medical knowledge database, presented and applicable data options selected. The algorithm conducts a statistical analysis of user-selected data options of each tier to determine numerical values indicating a likelihood that an associated ailment or condition is a cause of the symptoms. A machine learning model receives and further refines the numerical values. A report is presented setting forth refined numerical values and the ailment or condition associated with each one of the refined numerical values.

### CROSS REFERENCE TO RELATED APPLICATIONS

The present application claims priority under 35 U.S.C. 119(e) to the provisional patent application filed on Nov. 17, 2021 and assigned application No. 63/2808608. The contents of that application are incorporated herein.

### FIELD OF THE INVENTION

The present invention relates to examining animal health conditions for determining relationships between presented symptoms and causative ailments and conditions.

### BACKGROUND OF THE INVENTION

One of the most troubling and confusing situations that a pet owner faces is when to seek veterinary care for a pet that appears to be sick. The situation is compounded when the pet provides little detailed information as to whether or not it is suffering a serious ailment. Thus, the pet owner may take her pet to the veterinarian for a thorough check-up, resulting in an expense that may have been unnecessary. And in other cases, the pet owner neglects to take her pet to the veterinarian and the situation worsens, as in fact the pet was suffering a significant illness. The present invention attempts to provide some guidance to the pet owner as to the nature of the pet's current condition, its severity, and any necessary healthcare requirements guidance. With this information in hand, the owner can better decide whether and when to seek veterinary care.

### BRIEF DESCRIPTION OF THE DRAWINGS

FIG. 1 is a flowchart showing the principle steps of the present invention.

FIGS. 2-5 are exemplary data input screen shots.

FIGS. 6 and 7 depict output reports generated by the system of the present invention.

FIG. 8 depicts a computer system for implementing the invention.

### DETAILED DESCRIPTION OF THE INVENTION

The system of the invention, commercially referred to as an EquiVet Care™ Virtual Examination System and Tool, maintains a pet health profile and provides a structured and dynamically-directed examination system to maintain the animal in a best health condition. The tool is intended for use by pet owners and veterinarians to better understand and manage concerns about an animal's health. The system is typically utilized with a domestic animal or family pet, but the tool can also be used to collect and analyze health information from animals under the care of a vet, show/competition animals, and livestock.

The system comprises an input device for collecting animal health data and an algorithm that operates on the collected data. The algorithm analyzes and compares the information entered by the user with information in a medical knowledge database (MKD), generating preliminary information as to the health condition of the animal. The algorithm output is further analyzed in a machine learning (AI/ML) model that refines the algorithm output. The system then provides a results page advising the user as to the pet's condition and its severity, and suggesting next-steps to obtain advisable or necessary health care for the pet.

As used herein, the user may refer to the pet owner, a vet, or a casual observer of the p pet who is concerned with the pet's health condition. Within the first year or two of life, the user will likely be the pet owner.

As used herein, the following phrases have the indicated meaning:

“Data values” describe symptoms, categories, environmental conditions, etc. that are set forth in the medical knowledge database. Every piece of information within the medical knowledge database (MDK), other than the ailment variable itself, is referred to as a data value.

“Data options” refer to symptoms, categories, environmental conditions, etc. as displayed or presented to the user, with the user selecting from among the presented symptoms, categories, environmental conditions, etc., based on the animal's condition.

“Instances” refer to its literal definition, that is, occurrences or examples, referencing multiple occurrences of the same type of database relationship or link. Each occurrence of these relationships links two data values in the MKD so that, after the user selects a data value, the algorithm identifies the link and then presents the corresponding or linked data value to the user.

The system of the invention provides multiple advantageous components and features as described herein.

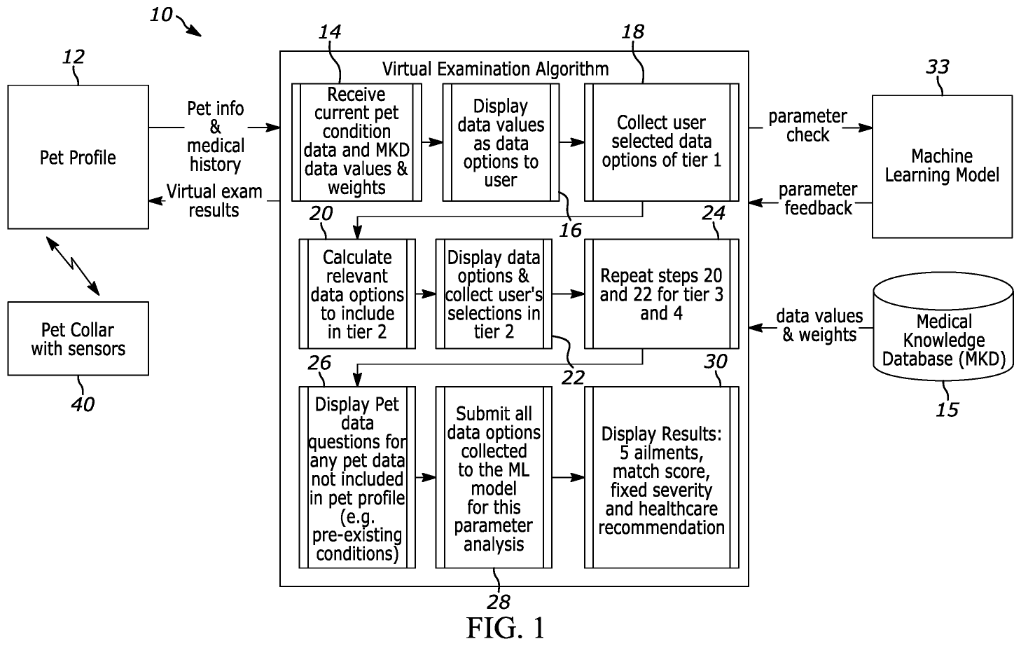

FIG. 1 illustrates a flowchart 10 describing steps associated with the present invention. Information from a pet profile 12 (a database) is supplied to the virtual examination algorithm (hereinafter algorithm). This information includes applicable medications, species, breed, age, weight, sex, pre-existing conditions, prior diagnoses, supplements, food intake and type, reproductive system alterations, location, recorded medical history, vaccines, dewormer, and treatments/therapies. All this information is important and relevant in determining the animal's present health condition. If a pet profile is available, system queries requesting such information can be omitted as that data can be collected directly from the profile.

The algorithm, which executes as the data is entered, is dynamic in deciding which data options to offer the user in response to data previously entered by the user. As the user completes one inquiry and continues to the next, the dynamic nature of the algorithm offers only those data options that are considered to be material to a final determination of the most likely presented ailment or condition.

At a step 14 of FIG. 1, the algorithm receives pet data from the pet profile 12 and also receives data from a medical knowledge database 15.

The medical knowledge database, operative in conjunction with the virtual examination algorithm, comprises data values and a weight associated with a relationship instance of each data value to an ailment or condition. As described above, the data values include symptoms, categories, environmental conditions, etc., that identify ailments and conditions of domestic animals, severity level indicators for each ailment, and health care suggestions or requirements (e.g., visit the local emergency room immediately) for each ailment.

Within the MKD, each ailment or condition is cataloged according to relevant categories of symptoms, the symptoms themselves, medications, environmental conditions, species, breed, age, weight, sex, pre-existing conditions, prior diagnoses, supplements, food intake and type, reproductive system alterations, exclusionary criteria, location, recorded medical history, vaccines, dewormer, treatments/therapies, indicators of an emergency condition, and exclusionary criteria inquiries.

Additionally, the medical knowledge database contains many instances of two relationships or links between data values. That is, a data value (e.g., symptom) may be indicative of two (or in some cases more than two) conditions or ailments. The relationships identify which data options populate for each inquiry or identify the categories, symptoms, pre-ex. conditions, etc. of each ailment, and weights for use in the virtual examination algorithm's calculations.

Each instance that identifies categories, symptoms, pre-exam conditions, etc. for each ailment or condition is assigned a numerical value (typically in percentage terms) indicating a level of confidence that the presented subordinate data value holds true for each related superordinate data value.

At step 16, a display or another presenting device presents the data values (information from the MDK database) as data options for user selection. The health data is collected as the user selects or answers each dynamic inquiry in the form of prompts or data options.

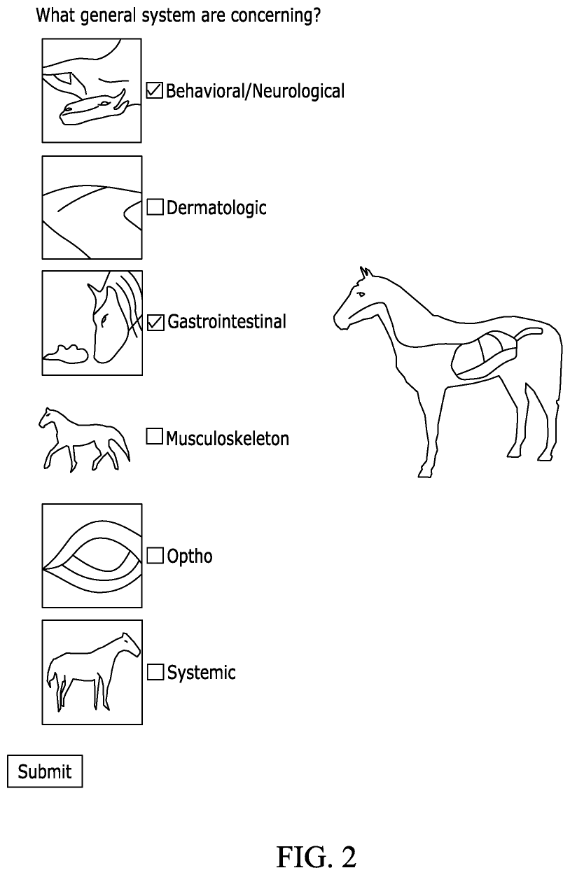

At step 18, the user responds to the presented data options by selecting applicable data options at a tier 1. Here the user identifies the bodily system(s) of concern, e.g., musculoskeletal, gastrointestinal, dermatological, ophthalmic, behavioral/neurological, and/or systemic. See FIG. 2 for an exemplary display (What general systems are concerning?) of presented data options and selections made by the user, i.e., behavioral/neurological and gastrointestinal as indicated by the checked boxes.

The health data provided as the user selects the data options and/or provides information includes both presently and historically observed conditions, both medically-related and circumstantial or situational. Typically, the data provided in response to the data options includes categories of symptoms, the symptoms themselves, medications, environmental conditions, species, breed, age, weight, sex, pre-existing conditions, prior diagnoses, supplements, food intake and type, reproductive system alterations, location, recorded medical history, vaccines, dewormer, treatments/therapies, indicators of an emergency condition, and exclusionary criteria inquiries. As described above, certain information can be obtained directly from the pet profile and is therefore not requested from the user by the algorithm.

At a step 20, the algorithm calculates relevant data options to include in a tier 2 inquiry. Typically, a tier 2 inquiry relates to either a subordinate or a superordinate class relative to a tier 1 inquiry, as determined by superordinate and subordinate tiers in the medical tree of the MKD.

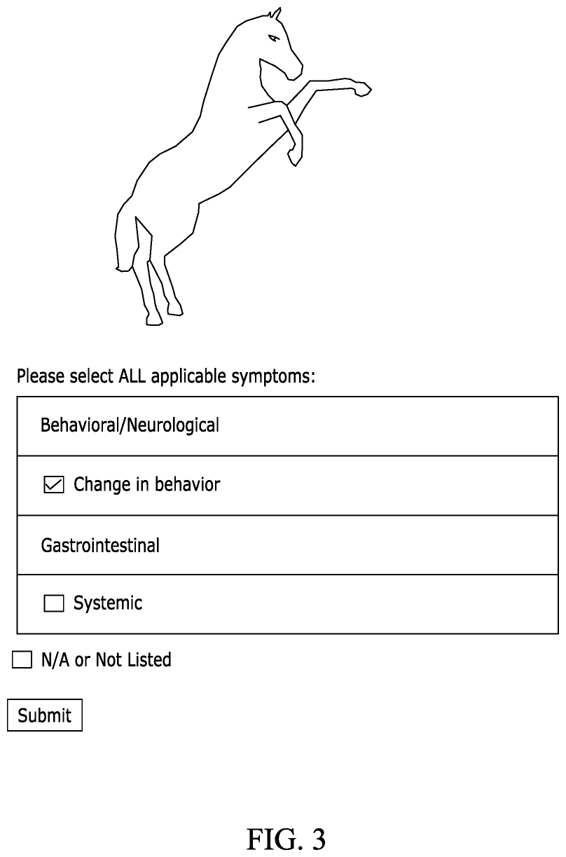

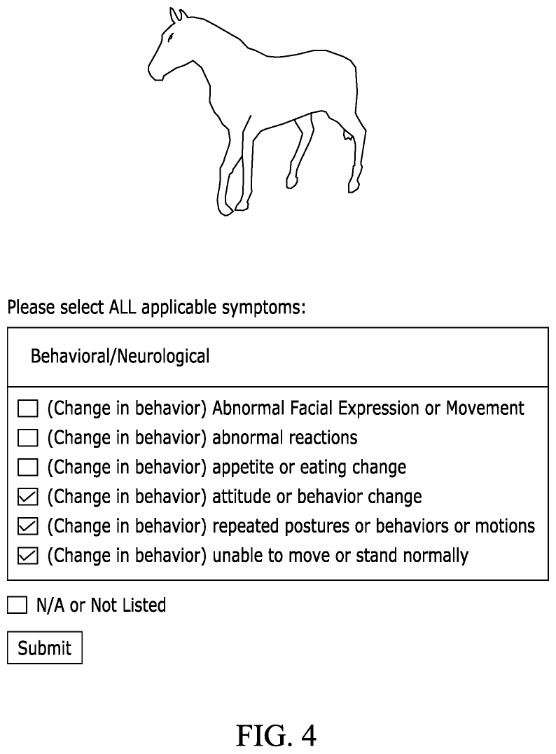

The tier 2 inquiry requires the user to further specify (that is, provide additional details/data option selections) their concerns by identifying the relevant or applicable categories of observable symptoms, conditions, and behaviors within each selected bodily system. See FIGS. 3 and 4.

For example, assume horse owner Penny had identified the bodily systems of concern for her horse as “musculoskeletal”, “dermatological”, and “behavioral/neurological” in the first or tier 1 inquiry. In the tier 2 inquiry, she selects the following data options in the musculoskeletal category; “abnormal movement”. For “dermatological,” Penny selects the data options “sweating”. For “behavioral/neurological” Penny selects “abnormal breathing,” “change in behavior,” and “abnormal food consumption.”

At step 22, the algorithm displays options and collects the user's selections in conjunction with the tier 2 inquiry.

Step 24 indicates that steps 20 and 22 may be repeated for additional tiers, such as tiers 3 and 4. Whether these additional steps are necessary is determined by the specific condition and the number of superordinate and subordinate classes that are relevant to each condition.

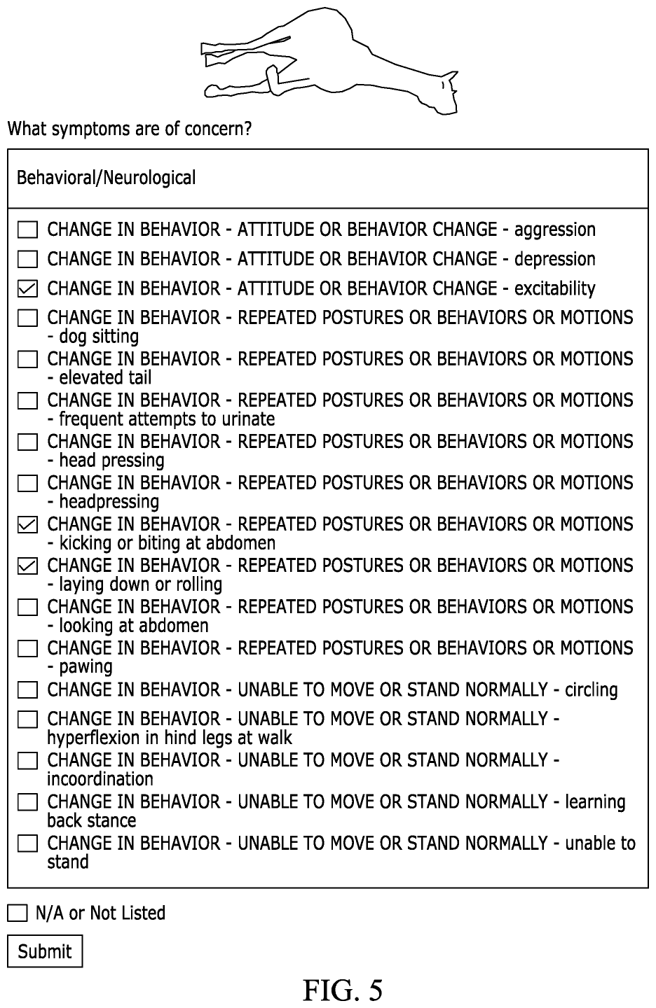

A tier 3 inquiry, for example, may require the user to identify details of the presently observable symptoms and behaviors as related to additional subordinate classes or to identify specific symptoms that are of concern. See FIG. 5. For example, after Penny makes the selections described above, she makes the following subordinate selections to further distinguish the concerns she has regarding her horse's health. For “abnormal movement” Penny selects “pawing the ground”. Penny's selection, “sweating”, cannot be further discerned as there exists no subordinate data value to “sweating” within the medical knowledge database. Note that subordinate data values are presented to the user to the extent such subordinate values exist in the MKD, as certain tiers may not subordinate data values.

For the tier 2 “abnormal breathing” Penny selects a subordinate condition, “rapidly breathing.” For “change in behavior” Penny selects “depressed”. For “abnormal food consumption” Penny selects “not eating”.

The level of inquiry detail (i.e., number of inquiry tiers) depends on the level or degree of differentiation provided by the data values in the medical knowledge database. As is evident from FIG. 1 and the accompanying description, the MKD is a critical element of the system and is consulted by the algorithm to elicit critical health data from the pet owner.

Whenever the medical knowledge database contains additional subcategories or sub-subcategories, the user is required to identify the applicable data options (as the system presents additional data values for additional tiers) among superordinate categories before continuing to identify the higher levels of detail in the subordinate categories.

At step 26, the system requests additional pet profile information from the user, such as pet profile information that might be relevant to a suspected current ailment, symptoms, or condition, if such information was not previously collected and is not available from the pet profile 12.

At a step 28, all the data collected, including the observed symptoms and behaviors, and the result obtained by the algorithm, are input to a machine learning algorithm or model 33, also referred to as an artificial intelligence/machine learning algorithm or model.

As can be seen from FIG. 1, the information from the virtual examination algorithm is input to the machine learning model 33, as indicated by an arrowhead and its label “parameter check”. The parameter check information includes the user data supplied to the algorithm as well as the algorithm results.

As with other machine learning models in use for other applications, the machine learning model of the present invention was previously trained to identify patterns of errors in the process of identifying and analyzing conditions/ailments/symptoms, to apply parameters to compensate for and correct any errors in the algorithm's identification of the most likely ailments or conditions, and to adjust the results of possible ailments and conditions and their scores accordingly.

The machine learning model 33 in FIG. 1 analyzes the input parameters and produces an output, referred to in FIG. 1 as “parameter feedback”. The virtual examination algorithm determines a relatively coarse list of possible medical ailments or conditions (diagnoses) based on a traditional medical knowledge database. With this information input to the machine learning model, the model can then generate a more precise list of possible diagnoses with a more accurate match score for each such condition. The machine learning model output is a more refined list of possible medical ailments or conditions with a score associated with each such condition or ailment. The model, and thus the resulting score is based on real-world experiences as reflected in in training dataset and can correct nuances that may have been missed by the algorithm.

Step 30 simply indicates that that the point values are displayed to the user. After step 28 has been executed all the data values collected from the user interaction have been analyzed by the medical knowledge database, virtual examination algorithm, and machine learning model to find all likely ailment and condition matches. Each of these likely ailment and condition matches is presented to the user as a point value that is related to the probability that the associated ailment or condition is responsible for the presented symptom. This information is compiled into a virtual examination health data report for the pet owner and presented to the user at step 30.

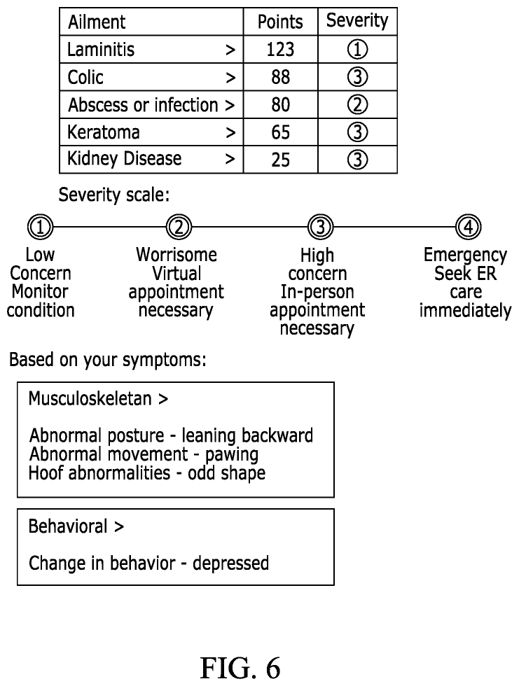

FIG. 6 depicts a report as generated by the system of the present invention. In one embodiment, the system (i.e., the algorithm operating in concert with the MKD and the separate machine learning model) displays a health data report indicating the top five ailments or conditions that are considered most likely responsible for the presented symptoms or behaviors. A match score indicates the likelihood that the associated ailment or condition is in fact a result of the symptoms or behaviors presented. The match scores are not presented as a percent, but rather as a raw score or number. Higher scores or values suggest a more likely cause for the identified symptom.

The report also indicates a level of ailment or condition severity (for example, that the presented condition is considered an emergency condition), reiterates all the user's identified data options (i.e., inputs), and provides guidance regarding a subsequent course of action (e.g., seek health care attention) to achieve the best possible outcome for the domestic animal.

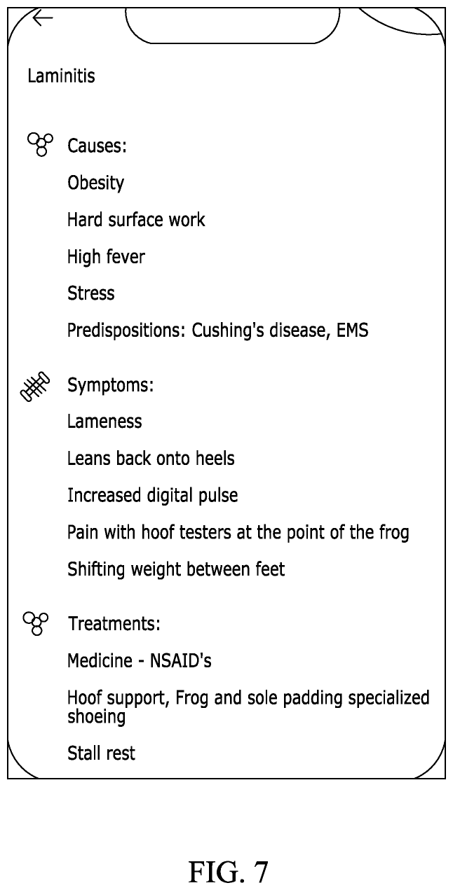

The last component of the report is depicted in FIG. 7. This FIG. 7 report is generated when the user selects or clicks on one of the conditions or ailments in FIG. 6. For the laminitis condition shown in FIG. 6, the subsequent report of FIG. 7 identifies the causes, symptoms and treatments.

This resulting/output health data report is incorporated into the pet profile as it serves as the domestic animal's central health-information repository, including all health and medical history information.

Returning to our Penny and her horse example, each data option that Penny selected represents a data value in the medical knowledge database that has a relationship or link: the “musculoskeletal” data value links to “abnormal movement”, the “abnormal movement” data value links to “pawing the ground.”

Also, each of these data values has a relationship or link with “colic”, that is colic is linked to “musculoskeletal”, “colic” is linked to “abnormal movement”, and “colic” is linked to “pawing the ground”. Each of these relationships has a value associated with it: for example, “colic” to “abnormal movement” has a value of 2 points, “colic” to “pawing the ground” also has a value of 2 points.

To complete the example;
“colic” as related to “abnormal movement” represents 2 points
“colic” as related to “pawing the ground” represents 2 points
“colic” as related to “sweating” represents 10 points
“colic” as related to “abnormal breathing” represents 8 points
“colic” as related to “rapidly breathing” represents 15 points
“colic” as related to “change in behavior” represents 8 points
“colic” as related to “depressed” represents 35 points
“colic” as related to “abnormal food consumption” represents 8 points
“colic” as related to “not eating” represents 13 points

The above point values identified for “colic” represent a correlation between the subordinate value and its superordinate value. Thus, a 2-point value for “pawing the ground” indicates that if this symptom alone is present, the likelihood that the horse has colic is represented by a 2 point value. Clearly far below the 35 point match with “depressed.”

As Penny selects the above data options, the virtual exam algorithm incorporates this data into its multivariate statistical analysis of variance, to produce the initial ailment matches that are then incorporated into the ML model along with the selected data options to refine the ailment matches according to the learned parameters of the ML model. In this case, the ML model concludes that the horse may have colic according to the presented match scores.

In conducting the statistical analysis to determine the most likely ailments based on user input, the algorithm performs a multivariate statistical analysis of variance (MANOVA) based on the medical knowledge database, each instance of the database's relationships, their assigned probability values, and all analyzed data. Basing the algorithm on these structures, allows the algorithm to calculate the variance of each data value within and between these structures to determine the statistical probability of the user's inputs relative to each possible ailment.

The system breaks down a hierarchical tree of high-level symptoms in the MDK. As the user responds to data options, the detail level becomes more descriptive of the ailments, the system matches this n-tier list of descriptions to possible ailments. Using this set of symptoms, the system combines additional information to calculate likely ailments. These additional descriptions include another tree of possible ailments within the MDK.

All this data is combined to generate a final list of possible ailments with a score based on all the parameters chosen.

Generally, there is no fixed or definitive relationship between points and percentages. Probabilities are additive based on the number of matching symptoms and matching categories. There can be ten symptoms per ailment, so there is overlap among ailments.

For example, during the virtual examination for Penny's horse, Penny selected the data options “pawing the ground”, “not eating”, and “depressed” worth 2 points, 13 points, and 35 points respectively. The algorithm will interpret and analyze this input data based on these structures to determine that there is a 50-points match that indicate that Penny's horse has colic.

The machine learning (artificial intelligence) model 33, has been trained by a dataset of animal symptoms and the determined diagnoses, and historical virtual examination algorithm results. As to training with algorithm, the machine learning model is trained on symptoms and previously determined diagnosis by the algorithm. This process allows the machine learning model to determine how the condition of the animal was interpreted by the algorithm and the accuracy of that determination, especially as compared to the diagnosis determined by the machine learning model. As indicated in FIG. 1, the machine learning model operates on output data from the algorithm.

A feedback loop implemented in the model allows the model to learn to conduct virtual examination analyses with greater medical/health accuracy and precision. This feedback loop feeds the algorithm's output back into the model's input, thereby conducting the analysis once again, effectively through an infinite loop. The feedback process allows the model to identify and predict patterns and conclusions from the data proactively and implement them into the virtual examination Al/ML model, thereby driving self-optimization of medical accuracy.

On element of the invention relates to exclusionary criteria, that is, the ability to eliminate certain potential health ailments or conditions by posing additional questions to the user before the system determines the most likely ailments or conditions. For example, Penny's horse presents the following symptoms; “change in behavior”, “rapidly breathing”, “sweating”. All of the listed symptoms are common in horses with colic, abdominal pain caused from various factors that can cause death if left untreated or are severe. However, certain ones of these symptoms may also indicate that the horse has just engaged in strenuous exercise, and needs to cool off with a walk, bath, and drinking water. Thus, the exclusionary criteria here is exercise, because if the horse had recently exercised, the inputted symptoms do not point to colic.

When the system presents Penny with multiple input options, she selects the three identified above. Her selections trigger an exclusionary criteria parameter within the MKD. This parameter prevents the misidentification of one ailment or condition for another by posing one or more additional inquiries to the user before completing the algorithm's analysis. The virtual examination system prompts Penny with a specific inquiry, asking if her horse had participated in strenuous exercise within the last ten minutes. After Penny replies “no” to this final inquiry, the virtual examination system concludes that Penny's horse may be experiencing colic. Note, the ten-minute interval is not to be construed as an absolute or definitive time frame, but ten minutes is generally sufficient time for a horse to cool down.

If Penny had responded “yes” to the inquiry, because the horse had exercised about ten minutes prior to the examination, then the system would have concluded that the horse is simply hot and needs to cool down. A colic condition would likely not be present.

The virtual examination algorithm of the present invention can be adapted to other situations that can advantageously use the algorithm to make critical evaluations. These situations are distinguished only by the data's subject matter. For example, the algorithm can be adapted to specific species, other than equine, feline, and canine animals, including other mammals, reptiles, amphibians, birds, and fish.

According to another embodiment, other health data collecting devices can be incorporated into the system of the invention. For example, a pet collar with appropriate sensor technology can collect data related to multiple different bodily conditions, such as heart rate. See a pet collar with sensors 40 in FIG. 1. This data can then be fed wirelessly to the pet profile repository of FIG. 1. Such historical data can be valuable when diagnosing a health condition of a pet using the system and tool of the present invention.

### Applications of the System and Tool

The virtual examination's output of health data results can be shared veterinarians, yielding improved care efficiency and effectiveness, expanding labor productivity, and client base experience and size.

After resolution of a case, the veterinarian can provide feedback on the accuracy of the virtual examination, to optimize the system output to correct identified issues, improve the algorithm's sensitivity, and improve the specificity of the analysis and determined condition.

Data provided by the virtual examination into the EquiVet Care System allows the EquiVet Care System to identify health trends or statistically significant symptoms or conditions as incidence levels changes. For veterinarians and animal health officials in need of identifying outlying health trends in a given geographical area, age group, breed, or species, the data allows for faster recognition and control efforts. Upon widespread adoption of the System, its data will present a map of domestic animal health in real time, providing a previously nonexistent perspective for stakeholders in animal health (e.g. clinical researchers, regulatory bodies, and insurance agencies).

### Computer System Description

The embodiments of the present invention may be implemented in the general context of computer-executable instructions, such as program modules executed by a computer. Generally, program modules include routines, programs, objects, components, data structures, etc. that perform particular tasks or implement particular abstract data types. For example, the software programs that underlie the invention can be coded in different languages for use with different platforms. The principles that underlie the invention can be implemented with other types of computer software technologies as well.

Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote computer storage media including memory storage devices.

Persons skilled in the art will recognize that an apparatus, such as a data processing system, including a CPU, memory, I/O, program storage, a connecting bus, and other appropriate components, could be programmed or otherwise designed to facilitate the practice of the method of the invention. Such a system would include appropriate program features for executing the method of the invention.

Also, an article of manufacture, such as a pre-recorded disk or other similar computer program product, for use with a data processing system, could include a storage medium and a program stored thereon for directing the data processing system to facilitate the practice of the method of the invention. Such apparatus and articles of manufacture also fall within the spirit and scope of the invention.

The present invention can be embodied in the form of computer-implemented processes and apparatuses for practicing those processes. The present invention can also be embodied in the form of computer program code containing computer-readable instructions embodied in tangible media, such as floppy diskettes, CD-ROMs, hard disks, flash drives or any other computer-readable storage medium, wherein, when the computer program code is loaded into and executed by a computer or processor, the computer or processor becomes an apparatus for practicing the invention. The present invention can also be embodied in the form of computer program code, for example, whether stored in a storage medium or loaded into and/or executed by a computer, wherein, when the computer program code is loaded into and executed by a computer or processor, the computer or processor becomes an apparatus for practicing the invention. When implemented on a general-purpose computer, the computer program code segments configure the computer to create specific logic circuits or processing modules.

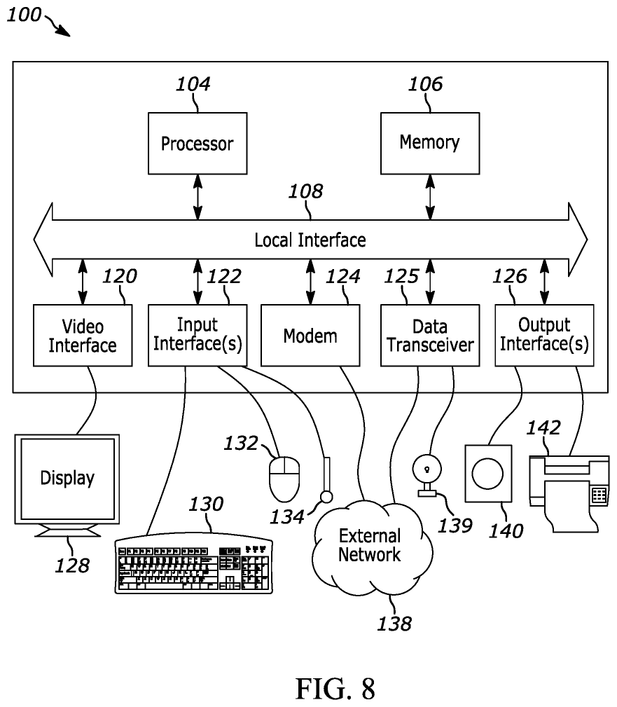

FIG. 8 illustrates a computer system 100 for use in practicing the invention. The system 100 can include multiple remotely-located computers and/or processors. The computer system 100 comprises one or more processors 104 for executing instructions in the form of computer code to carry out a specified logic routine that implements the teachings of the present invention. The computer system 100 further comprises a memory 106 for storing data, software, logic routine instructions, computer programs, files, operating system instructions, and the like, as is well known in the art. The memory 106 can comprise several devices, for example, volatile and non-volatile memory components further comprising a random access memory RAM, a read only memory ROM, hard disks, floppy disks, compact disks including, but not limited to, CD-ROM, DVD-ROM, and CD-RW, tapes, flash drives and/or other memory components. The system 100 further comprises associated drives and players for these memory types.

In a multiple computer embodiment, the processor 104 comprises multiple processors on one or more computer systems linked locally or remotely. According to one embodiment, various tasks associated with the present invention may be segregated so that different tasks can be executed by different computers located locally or remotely from each other.

The processor 104 and the memory 106 are coupled to a local interface 108. The local interface 108 comprises, for example, a data bus with an accompanying control bus, or a network between a processor and/or processors and/or memory or memories. In various embodiments, the computer system 100 further comprises a video interface 120, one or more input interfaces 122, a modem 124 and/or a data transceiver interface device 125. The computer system 100 further comprises an output interface 126. The system 100 further comprises a display 128. The graphical user interface referred to above may be presented on the display 128. The system 100 may further comprise several input devices (not shown) including, but not limited to, a keyboard 130, a mouse 131, a microphone 132, a digital camera and a scanner (the latter two not shown). The data transceiver 125 interfaces with a hard disk drive 139 where software programs, including software instructions for implementing the present invention are stored.

The modem 124 and/or data receiver 125 can be coupled to an external network 138 enabling the computer system 100 to send and receive data signals, voice signals, video signals and the like via the external network 138 as is well known in the art. The system 100 also comprises output devices coupled to the output interface 126, such as an audio speaker 140, a printer 142, and the like.

While the invention has been described with reference to preferred embodiments, it will be understood by those skilled in the art that various changes may be made and equivalent elements may be substituted for elements thereof without departing from the scope of the present invention. The scope of the present invention further includes any combination of the elements from the various embodiments as set forth herein. In addition, modifications may be made to adapt the teachings of the present invention to a particular application without departing from its essential scope. Therefore, it is intended that the invention not be limited to the particular embodiment disclosed as the best mode contemplated for carrying out this invention nor to the other embodiments described and/or illustrated, but that the invention will include all embodiments falling within the scope of the appended claims.

Although the subject matter of the invention has been described in relation to specific structural features and/or acts, it is to be understood that the subject matter defined in the appended claims is not necessarily limited to the specific features or acts described above. Rather, the specific features and acts described above are disclosed as example forms of implementing the claims. Accordingly, the invention is not limited except as by the appended claims.

What is claimed is:

1. A method for identifying one or more possible ailments or conditions as a cause of observed health-related symptoms of an animal, the method comprising:
retrieving information from a pet profile database comprising medical information and a medical history of the animal;
receiving observed health-related symptoms of the animal;
presenting first tier data options to a user, the first-tier data options related to a first tier of inquiry regarding the symptoms;
receiving applicable first tier data options from among presented first tier data options;
an algorithm for analyzing the applicable first tier data options using information from a medical knowledge database, information from the pet profile database, and observed symptoms;
wherein data options associated with each successive tier are determined by the algorithm based on user-selected responses to data options of a prior tier, information from the medical knowledge database, information from the pet profile database, and observed symptoms, wherein each tier is subordinate or superordinate relative to a preceding tier
presenting data options associated with each successive tier to the user;
from the user, receiving data options for each tier from among presented data options for each tier;
the algorithm conducting a statistical analysis of user-selected data options of each tier based on information from the medical knowledge database, information from the pet profile database, and observed symptoms, the statistical analysis determining a numerical value indicating a likelihood that an associated ailment or condition is a cause of the symptoms;
after all tiers of inquiry have been completed, inputting the numerical values to a machine learning model;
the machine learning model generating refined numerical values, each refined numerical value associated with an ailment or condition and indicating a likelihood that an associated ailment or condition is a cause of the symptoms; and
presenting a plurality of the refined numerical values and the ailment or condition associated with each one of the plurality of refined numerical values.

2. The method of claim 1, further comprising presenting a severity indicator for each ailment or condition, a healthcare recommendation for each ailment or condition, and healthcare information for each ailment or condition.

3. The method of claim 1, further comprising adding to the pet profile database the refined numerical values and the ailment or condition associated with each one of the refined numerical values.

4. The method of claim 1, wherein the plurality of refined numerical values comprises a top five refined numerical values and the ailment or condition associated with each one of the top five refined numerical values.

5. The method of claim 1, wherein one of the symptoms comprises a current or an historical symptom.

6. The method of claim 1, wherein a symptom relates to animal behavior.

7. The method of claim 1, wherein the information from the pet profile database comprises one or more of, species, breed, age, weight, sex, pre-existing health conditions, prior diagnoses, environmental conditions, relevant medications, reproductive system alterations, supplements, food intake and type, location, recorded medical history, vaccines, dewormer, and treatments/therapies.

8. The method of claim 1, wherein a step of presenting first tier data options comprises presenting the first-tier data options on a display.

9. The method of claim 1, further comprising determining real-time symptoms of the animal using a remote monitoring sensor in contact with a body of the animal.

10. The method of claim 1, further comprising determining real-time health related parameters of the animal using a remote monitoring sensor, wherein a real-time health parameter comprises heart rate.

11. The method of claim 1, wherein real-time symptoms of the animal are stored in the pet profile database.

12. The method of claim 1, wherein a first-tier comprises bodily systems further comprising musculoskeletal, gastrointestinal, dermatological, ophthalmic, behavioral/neurological, and systemic, and wherein a second tier comprises symptoms associated with one or more of the bodily systems.

13. The method of claim 1, wherein information in the medical knowledge database is presented in tiers, wherein each tier is subordinate or superordinate relative to a previous tier.

14. The method of claim 13, wherein data values in a tier are related to data values in another tier by weight values.

15. The method of claim 13, wherein a second tier is subordinate to a first tier, and wherein each second-tier data value is related to a superordinate data value according to a numerical value.

16. The method of claim 1, wherein the pet profile database serves as a training dataset for the machine learning model.

17. The method of claim 1, wherein certain health-related conditions are location-related, and wherein determining that the animal has not visited a certain location excludes location-related conditions associated with the certain location as a cause.

18. The method of claim 1, wherein the algorithm comprises weights for use in the statistical analysis for determining a numerical value indicating a likelihood that an associated ailment is a cause of the symptoms or conditions.

19. A system for implementing the method of claim 1.

20. A system for identifying one or more possible ailments or conditions as a cause of observed health-related symptoms of an animal, the system comprising:
at least one processor; and
at least one memory including one or more sequences of instructions, the at least one memory and the one or more sequences of instructions configured to, with the at least one processor, cause the system to perform at least the following:
a. retrieving information from a pet profile database comprising medical information and a medical history of the animal;
b. receiving observed health-related symptoms of the animal;
c. presenting first tier data options to a user, the first-tier data options related to a first tier of inquiry regarding the symptoms;
d. receiving, from a user applicable first tier data options from among presented first tier data options;
e. analyzing the applicable first-tier data options using information from a medical knowledge database, information from the pet profile database, and observed symptoms;
f. determining data options associated with each successive tier based on user-selected responses to data options of a prior tier, information from the medical knowledge database, information from the pet profile database, and observed symptoms, wherein each tier is subordinate or superordinate relative to a preceding tier
g. presenting data options associated with each successive tier to the user;
h. receiving data options for each tier from among presented data options for each tier;
i. conducting a statistical analysis of user-selected data options of each tier based on information from the medical knowledge database, information from the pet profile database, and observed symptoms, the statistical analysis determining a numerical value indicating a likelihood that an associated ailment or condition is a cause of the symptoms;
j. after all tiers of inquiry have been completed, inputting the numerical values to a machine learning model;
k. the machine learning model generating refined numerical values, each refined numerical value associated with an ailment or condition and indicating a likelihood that an associated ailment or condition is a cause of the symptoms; and
l. presenting a plurality of the refined numerical values and the ailment or condition associated with each one of the plurality of refined numerical values.
